(19) United States
(12) Patent Application Publication
(10) Pub. No.: US 2016/0000395 A1
(43) Pub. Date: Jan. 7, 2016

(54) Assessing Optical Density Gradients and Variations

(71) Applicant: MARIKA PTY LTD, Western Australia (AU)

(72) Inventors: Abbey John Perumpanani, Korora (AU); Joan Arakkal, Bateman (AU)

(73) Assignee: MARIKA PTY LTD, Bateman, WA (AU)

(21) Appl. No.: 14/769,556

(22) PCT Filed: Mar. 4, 2014
(86) PCT No.: PCT/IB2014/059413

§ 371 Date: Aug. 21, 2015

(30) Foreign Application Priority Data
Mar. 6, 2013 (AU) 2013900776
May 28, 2013 (AU) 2013901902

(51) Int. Cl. A61B 6/00; G06T 7/00; A61B 8/08; A61B 5/00; A61B 5/055

(52) U.S. Cl. CPC A61B 6/505; A61B 5/4504; A61B 5/4509; A61B 5/055; A61B 6/5217; A61B 8/0875; A61B 8/5223; G06T 7/0012; G06T 2207/10004; G06T 2207/30008

(57) ABSTRACT

The invention relates to a method of assessing an optical density gradient in one or more radiologic images of a non-uniformly composed material for purpose of using the optical density gradient to assess a characteristic of the non-uniformly composed material. Optical density gradient is calculated as a function of the difference in optical density between a first and second region on the one or more images. The method finds particular application in predicting occurrence or likelihood of non-union of a bone fracture and for predicting occurrence of likelihood of a disorder or disease of the bone, such as osteoporosis.

### FIELD OF THE INVENTION

The present invention relates to assessment of optical density gradient and/or optical density variations in images, such as radiologic images, of a non-uniformly composed material, including in the human or animal body.

The present invention finds particular application in determining occurrence or likelihood of complications in the healing process of a bone fracture, such as non-union.

The present invention also finds particular application in following up the progress of the healing process of a bone fracture, such as estimating the end point of treatments like fracture immobilisation.

The present invention also finds application as a diagnostic and screening tool of bone conditions, such as osteoporosis.

### BACKGROUND TO THE INVENTION

In recent years there have been significant developments in management of bone fractures through improved imaging techniques and early intervention methods. Together, these advances can significantly reduce incidence of complications and can help to reduce the overall period of immobilisation required for the fracture to heal appropriately.

Despite such advances, difficulties can sometimes still be experienced in appropriately managing fractures. In most cases, bone fractures can be treated by returning fractured bone to its natural, pre-fracture position and immobilising the bone in this position while the bone undergoes its natural healing process to knit the fracture ends together.

Some fractures, however, may require surgical intervention to assist the healing process or ensure that healing occurs. Often, surgical methods are undertaken only if immobilisation methods have failed, are predicted likely to fail or will likely result in poor functionality of the affected area. It can be difficult to determine when surgery or other interventions are appropriate. For example, it may not be apparent that a bone fracture initially treated with immobilisation treatment actually requires surgical or other intervention until such time as the cast or splint is removed and functionality assessed, which is usually at least some weeks after the injury occurred. If surgical intervention is needed at this time, not only does it increase the total treatment and healing time significantly, but delay in healing can of itself lead to complications. Typical complications of fracture healing include delayed union, non-union or fibrous union of the fracture site.

Some types of fractures in particular may be more prone to complications than others. One example is in the management of scaphoid fractures. The proximal part of the scaphoid is entirely dependent on intra-osseous blood supply and fractures can disrupt this blood supply, thereby compromising the healing process, since poor blood supply is a common contributor to delayed or failed union of the fracture.

It is estimated that non-unions occur in approximately 5-10% of all scaphoid fractures, with the incidence being higher in displaced fractures. Various studies have documented progression of non-union to collapse and arthritis.

The traditional approach to scaphoid fracture treatment is, as with most other fracture types, immobilisation if an x-ray radiograph shows presence of a fracture. Scaphoid fractures typically require immobilisation for about 12 weeks, or even longer, if, for example, the fracture is located more proximally. If there is clinical suspicion that a fracture has occurred despite normal initial x-ray imaging, the wrist is immobilised and x-ray imaging repeated about two weeks following the injury to determine whether the fracture has become visible due to lucency developing around the fracture site. This approach has been modified somewhat in recent times due to concerns about unnecessary or prolonged periods of immobilisation and its associated consequences and also due to occurrence of non-union despite apparently adequate immobilisation.

In the case of scaphoid fractures, the majority of cases were traditionally treated initially with immobilisation, and later through one or more operations (surgical intervention) if complications such as non-union arose despite a period of immobilisation. Globally, this continues to be the preponderant approach.

However, there now appears to be an emerging trend towards acute operative treatment when fractures can be seen on an initial x-ray image. If no fractures are visible, yet clinical suspicion of fracture injury persists, magnetic resonance imaging (MRI) is conducted. Following this assessment, proximal pole fractures visible with the MRI are treated operatively with all others being treated with immobilisation. Follow-up assessment, such as in the form of a computed tomography (CT) scan, is typically conducted about six weeks following injury. If this shows signs of healing in the form of disappearance of the fracture line, spot welding or callus formation, then a gradual return to activity is usually permitted. If there is an absence of any of these signs of healing, a CT scan is desirably repeated about four to six weeks later. If non-union is still apparent, the fracture is treated operatively, such as by a bone graft.

These assessment methodologies and subsequent treatment schedules, such as outlined above, have contributed to significantly improving the clinical outcomes for more difficult types of fractures such as scaphoid fractures. With operative treatment, union rates typically approach 95%. Use of MRI scans assists patients who do not show visual signs of fracture on an initial x-ray despite clinical suspicion of fracture, to avoid unnecessary periods of immobilisation.

However, despite obvious improvements in assessment and treatment schedules, several problems still persist. In the case of scaphoid fractures treated without an initial operation, but simply with immobilisation, as per the traditional approach, about 5% of patients will experience non-union of the fracture. It can take up to twelve weeks of immobilisation before a definitive diagnosis of non-union is made. Following definitive diagnosis, operative treatment is then required, which leads to several more months of immobilisation and the associated physical restrictions that are experienced as a result of this immobilisation.

In the case of scaphoid fractures treated through acute operative treatment, as per the emerging trend, a significant proportion (about 95%) of patients whose fractures would have healed without surgical intervention are being treated with an operation in order to prevent the complication of non-union in a small proportion (about 5%) of patients. While operations are becoming increasingly safe, they entail surgical risks and costs.

There is therefore a need for capability to make an accurate assessment of whether or not a fracture is at risk of non-union or improper union as early as possible following injury. Such a capability would allow non-operative treatment through immobilisation of those deemed at low risk of non-union, and seeking recourse to surgical treatment in only those deemed at high risk of non-union, either at the outset or as the immobilisation progresses, Therefore assessment and treatment schedule for fracture injuries would benefit from having ability to predict occurrence or likelihood of non-union of the fracture. To date, there is no reliable and definitive way of making such a predictive assessment of likelihood of occurrence of non-union.

Another problem in the treatment of fractures is determining the point in time at which a fracture can be deemed to have satisfactorily healed. After this time, treatment measures such as immobilisation can be ceased and patients allowed a gradual return to their work and activities of daily living. Current approaches to determining the total period of immobilisation for each patient is based on estimates of the upper limit of the period needed for fracture healing in a cohort of similar fractures. As a result, a number of patients needlessly undergo longer immobilisation than the minimum needed. Such immobilisation can delay return to regular activity. In some patients, this delay can be detrimental to their work and/or capacity to earn.

There is therefore a need for a capability to assess the progress of the fracture healing process in order to accurately determine when the fracture can be deemed to have healed. Such a capability will aid determination of when a treatment modality such as immobilisation of the fractured bone can be ceased and the patient allowed to mobilise the injured area thereby enabling return to the daily living and work activities.

Another type of pathology that is problematic, both in terms of diagnosis, treatment and follow-up, is osteoporosis. Osteoporosis is well recognised as a progressive systemic skeletal disease, characterised by reduced bone density and deterioration of bone tissue. The disease occurs when bones lose minerals, such as calcium, more quickly than the minerals can be replaced by the body. This loss of minerals leads to a loss of bone density. As bones lose density, minor trauma and accidents, such as a relatively minor fall, can cause serious fracture of the bone. When a patient presents with such injury, their bone density will generally already be so diminished as to be categorised as established osteoporosis.

The process that leads to established osteoporosis, is asymptomatic and the established condition usually only presents or is recognised after such serious bone fracture occurs. By the time such serious fractures occur, onset of osteoporosis has already begun, in some cases having become well advanced. In those affected, fractures can lead to a variety of complications including chronic and substantial pain, general disability and loss of mobility and in some cases even death.

Primary care should therefore ideally attempt to identify patients at increased risk before symptoms develop so that they may be offered appropriate treatment and/or preventative measures such as bone-preserving agents or implementation of revised diet and exercise regimes. Currently, osteoporosis is typically diagnosed with a bone density scan, also known as bone densitometry or bone mineral density (BMD) scan, which measures the density of bones. BMD is measured as it generally correlates with bone strength and hence ability to bear weight and cope with trauma.

A commonly used type of BMD scan is the dual energy x-ray absorptiometry (DXA) scan. DXA scans are considered the gold standard in diagnosis of osteoporosis but scans are often only considered once a patient presents with serious injury or has been identified as at serious risk using other indicators.

Measurements made using DXA scans are compared to the normal range for someone of the same ethnicity and its interpretation is hence dependent on a clear identification of the ethnicity of the subject. Further, current scanning techniques have limited capacity to monitor progression of osteoporosis or accurately measure whether or not treatment steps are having desired effect. There is some evidence to suggest that interval monitoring using scans such as DXA may be of limited value. Since bone density usually changes slowly, the actual physical changes may be smaller than the measurement error of the scanning machine. A repeat scan, at a one or two year interval, may not be able to distinguish between real density change or variation in measurement. In addition, DXA measures are dependent on body composition and hence longitudinal studies in persons who undergo significant changes in body composition, such as putting on weight, can be biased. Furthermore, since scanning usually is conducted on select bones, it may not be able to provide reliable information on differing rates of progression of the disease in different bones nor of. The present invention seeks to remedy or at least alleviate these deficiencies.

It is known that the radiologic images of osteoporotic bones shows greater lucency compared to normal bones. Evidence of this can often be seen in the large number of radiologic investigations done on patients in the risk group for osteoporosis for reasons other than the investigation of osteoporosis. Despite the availability of such a large number of images, and the ease with which they can be carried out, it is currently not possible to leverage these images to stratify the risk of osteoporotic fractures because of variations in radiographs due to quality, technique and exposure.

It would be useful if a high risk of osteoporosis, and therefore of osteoporotic fractures, can be diagnosed by a method that can be applied to radiologic images, regardless of the reason for having taken the radiologic image.

### SUMMARY OF THE INVENTION

According to a first aspect of the present invention there is provided a method of assessing an optical density gradient in one or more radiologic images of a non-uniformly composed material, the method comprising:
a) determining optical density (OD) of at least a first region and a second region of the one or more radiologic images;
b) determining distance between the first and second regions,
c) calculating the optical density gradient as a function of the optical densities of the first and the second regions and the distance therebetween; and
d) using the optical density gradient to assess a characteristic of the non-uniformly composed material.

Preferably the radiologic image is an image taken using imaging technology capable of exhibiting variations in density, desirably optical density. Non-limiting examples of radiologic images are x-ray, computed tomography (CT), magnetic resonance image (MRI), ultrasound or positron emission tomography (PET).

The present invention finds particular application in assessment of at least one characteristic of bone, including at least one characteristic of bone that has been injured to create a fracture. However, the present invention finds application in assessment of other materials and also in bones without fractures having a non-uniform composition.

Preferably, optical density is determined for a plurality of regions R1 to Rx, where x is the total number of measurement regions from which an optical density reading has been taken on the image or images being assessed, corresponding to the non-uniformly composed material. Each measurement region is marked out on the image at substantially regular intervals along at least one predetermined axis of a portion of the material. Size and shape of the measured regions and the distance therebetween are determined with reference to the nature of the material and the characteristic of the material that is being assessed.

Optical density is determined for at least one reference region, the or each reference region being marked out on a portion of the image or on another image. It is preferred that the size and shape of the reference region is substantially the same as the other measured regions for the assessment of any one image. An average optical density of the or each reference region, typically the mean of at least three separate optical density measurements, is determined to account for variable exposure and penetration.

Optical density of each region is measured by any suitable means, including appropriate software. Preferably, optical density is measured using the Region of Interest (ROI) tool on a picture archiving and communication system (PACS). The average or mean of such measured optical densities are conveniently referred to as raw optical density values.

It is preferred that any raw optical density measurements taken of a region of the image corresponding to the material are standardised to account for differences in, for example, exposure, particular specifications of the machine used to take the image and to eliminate effects of background ‘noise’. In a preferred embodiment, optical density measurements are standardised by determining difference(s) between the reference measurement and the measurement of regions on the image corresponding to the material. The resulting standardised optical density values may be conveniently referred to as S values. A series of standardised optical density values can be represented by the values S1 to Sx, where x is the total number of measurement regions from which an optical density measurement has been taken on the image. In a preferred embodiment, optical density measurements are standardised against at least one optical density measurement of an area that is different from the already measured regions.

Following standardisation of optical density measurements, each standardised OD measurement (S1 to Sx) is normalised against a selected standardised OD measurement. Preferably, normalised optical densities are calculated by normalising the standardised optical density measurements against one of the standardised optical density measurements. The normalised OD measurements can be conveniently referred to as N1 to Nx where x is the total number of measurement regions from which an optical density reading has been taken on the image or images being assessed.

Normalised OD values are conveniently and advantageously used to determine optical density gradients, which may quantify a quality of the non-uniformly composed material. Preferably, an optical density gradient is calculated between every possible pairing of N1 to Nx. The optical density gradient (G) is calculated as a function of the distance between the N values in every pair. This may conveniently be represented by the following equation:

G_{i,j}=(function of the Normalised Optical densities Nᵢ and Nⱼ)/(function of the distance between the regions where Nᵢ and Nⱼ)

where i≠j, and i.j are any numbers ranging from 1 to x and where x is the total number of measurement regions from which an optical density reading has been taken on the image being assessed.

In a preferred embodiment, this is calculated as

G_{i,j}=(Nᵢ−Nⱼ)/(distance between and Rⱼ)

It is preferred that distance between the regions yielding Nᵢ and Nⱼ is measured with reference to the same location on each measured region. For example, distance between respective centres of the measured regions, or between respective proximal sides of the measured regions.

Preferably, the optical density of a plurality of regions on the radiologic image may be measured along a section of the image of the material and an optical density gradient is determined between each pair of optical density measurements. One or more of the normalised optical density values and/or the optical density gradients can be used to assess at least one characteristic of the non-uniformly composed material.

It should be understood that one or more of the regions may be two dimensional or three dimensional. That is, each region may correspond to an area or a volume of the non-uniformly composed material.

In a preferred embodiment, each of the calculations giving rise to the normalised optical density and/or optical density gradient for each pairing of normalised optical density values is performed automatically. It is desirable if the calculations can be performed as a function of software used in determining the initial raw optical density values.

One or more forms of the present invention find(s) particular application in assessment of bone and more particularly in the assessment of a fracture site on the bone. Advantageously, the present invention can be employed in assessing the likelihood or occurrence of non-union of a bone fracture.

One or more forms of the present invention finds particular application in determining occurrence or likelihood of non-union of the fracture by assessing an optical density gradient in a radiologic image across a suspected bone fracture.

Thus, according to a further aspect of the present invention, there is provided a method of predicting occurrence or likelihood of non-union of a bone fracture, the method comprising:
a) determining optical density of at least a first region and a second region of a radiologic image corresponding to the bone;
b) determining distance between the first and second regions;
c) determining an optical density gradient between the first and second regions as a function of difference in optical density between the first and second regions and the distance therebetween;
whereby, an optical density gradient greater than a predetermined value is deemed indicative of likelihood or occurrence of non-union of the fracture.

It is preferred that prior to determining distance between the first and second regions, the optical density readings are first standardised and then normalised. Optical density gradient between first and second regions can then be determined as a function of difference in normalised optical density between the first and second regions and the distance therebetween.

It is preferred that an average optical density is determined for each region, typically comprising the mean of at least three separate optical density measurements.

The predetermined value which indicates likelihood or otherwise of non-union is determined at least partially on the basis of variables such as the type of bone being assessed.

It is preferred that optical density gradient relating to a fracture or suspected fracture is determined over a period of time.

It is preferred that a first optical density gradient is determined on or shortly after injury leading to the fracture or suspected fracture and, preferably, on at least one more occasion, some time after date of injury. The length of the time or times after injury that optical density gradient is subsequently determined is at least partially dependent on variables such as the type of bone, type of fracture and specific purpose for which the optical density gradient is being determined. Typically, optical density gradient is determined one or more times between day 6 and 100 following the injury.

According to one or more embodiments, variations in the optical density gradient over time may be used to make a prediction as to when the fracture will reach a clinically accepted point of adequate fracture healing, such as when optical density gradient will fall below a predetermined minimum value.

According to one or more embodiments, the optical density gradient may be used to determine when the fracture has reached a clinically accepted point of adequate fracture healing, such as when the optical density gradient has fallen below a predetermined minimum value.

One or more forms of the present invention also finds particular application in detecting disorders of bone, such as local and/or systemic osteoporosis by assessing a normalised optical density and/or an optical density gradient in one or more images of one or more bones from a person suspected of being affected by such a disorder.

Thus, according to yet a further aspect of the present invention, there is provided a method of predicting occurrence or likelihood of a disorder or disease of bone, the method comprising:
a) determining optical density of at least a first region and a second region in one or more radiologic images corresponding to bone;
b) determining an optical density gradient between the first and second regions as a function of difference in optical density between the first and second regions and the distance therebetween;
whereby an optical density gradient greater than a predetermined value is deemed indicative of likelihood or occurrence of a bone disorder.

Again, it is preferred that prior to determining distance between the first and second regions, the optical density regions are first standardised then normalised. Normalised optical density reading and/or optical density gradient greater than a predetermined value is deemed indicative of likelihood or occurrence of a bone disorder.

In a preferred embodiment, the bone disorder is osteoporosis and the method provides a means for diagnosing and following up on local and/or systemic osteoporosis by detecting exaggerated normalised values and/or gradients between different parts of one or more bones suspected of being affected. The method can also be usefully applied to detect variations in density throughout the bone and also to detect micro-fractures.

In one embodiment, the first and second regions of the radiologic image correspond to portions of the same bone. In a further embodiment, the first and second regions of the radiologic image correspond to portions of two different bones, enabling comparison of optical densities between the two bones. This finds practical application in assessing differential rate at which a bone disorder, such as osteoporosis, affects or is affecting different bones at any point in time. In this embodiment, it is not necessary to measure distance between the regions. Assessment can be made on the premise of comparison between the normalised OD of each region and similarly measured values from a ‘normal’ subject.

### BRIEF DESCRIPTION OF THE DRAWINGS

To assist with understanding the invention, reference will now be made to the accompanying drawings in which:

FIG. 1 shows a schematic representation of a radiologic image of a bone fracture with reference areas marked thereon;

FIG. 2 is a radiologic image of a scaphoid bone with multiple optical density reference areas marked thereon;

FIG. 3 is a graphical representation of optical density measurements along a normal (uninjured) scaphoid bone, the optical density measurements determined according to the method of the present invention;

FIG. 4 is a graphical representation of optical density measurements along a fractured scaphoid bone, the optical density measurements determined according to the method of the present invention;

FIG. 5 is a graphical representation of optical density measurements along a fractured scaphoid bone, the optical density measurements determined according to the method of the present invention and illustrating fluctuation in optical density over time;

FIG. 6 is a graphical representation of analysis of mean optical density gradients, differentiating between fractures progressing to union and those demonstrating non-union.

### DETAILED DESCRIPTION OF AN EMBODIMENT OF THE INVENTION

The following description and example of the present invention are made with particular reference to application of the method in assessing and quantifying optical density gradients of x-ray radiologic images taken of actual or suspected bone fractures. Particular examples are given in respect of application of the method in assessing or predicting occurrence or likelihood of non-union of a fracture of a scaphoid bone. However, the present invention is not limited to such a narrow range of application. It should be understood that the method may be successfully employed in such assessment of all types of bone fractures.

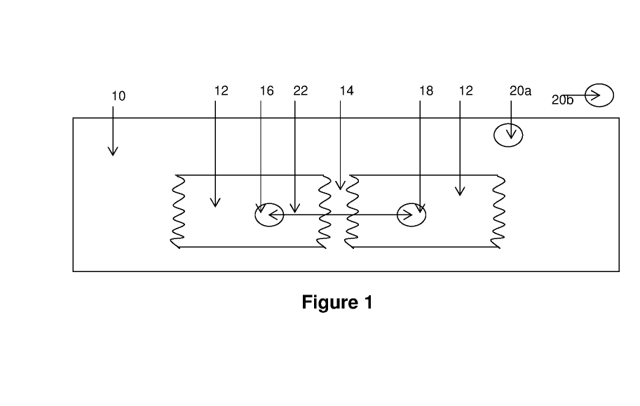

Referring initially to FIG. 1, there is shown an image 10, in this case a radiologic image, namely an x-ray image, of at least a portion of a material for which it is desired to assess a quality thereof, in this case a bone 12 which has experienced injury leading to fracture 14. FIG. 1 illustrates a complete fracture of the bone 12. However, the fracture 14 may be of any type.

The image 10 is typically a radiologic image such as an x-ray and the following description and examples illustrate application of the method in relation to an x-ray image. However the image 10 may be produced from any type of imaging technology capable of exhibiting variations in density, desirably optical density, of the material to be assessed, such as an image from computed tomography (CT scan), magnetic resonance imaging (MRI), ultrasound, positron emission tomography (PET) or other medical imaging techniques. It is this image, which may be in a digital or hard copy format, from which optical density (OD) measurements are taken, processed and assessed in accordance with the present method.

Referring still to FIG. 1, at least a first measurement region 16 and a second measurement region 18 having a distance 22 therebetween, are located on an area of the image 10 corresponding to the bone 12. The first measurement region 16 is located on one side of the fracture 14 and the second measurement region 18 is located on an opposing side of the fracture 14. The first measurement region 16 is desirably located on a superior side of the fracture 14 or, if that cannot be determined, the anterior side and if neither of those can be determined, the side of the fracture 14 that is lateral. The first measurement region 16 and second measurement regions 18 are located on respective portions of the bone 12 on opposing sides of the fracture 14.

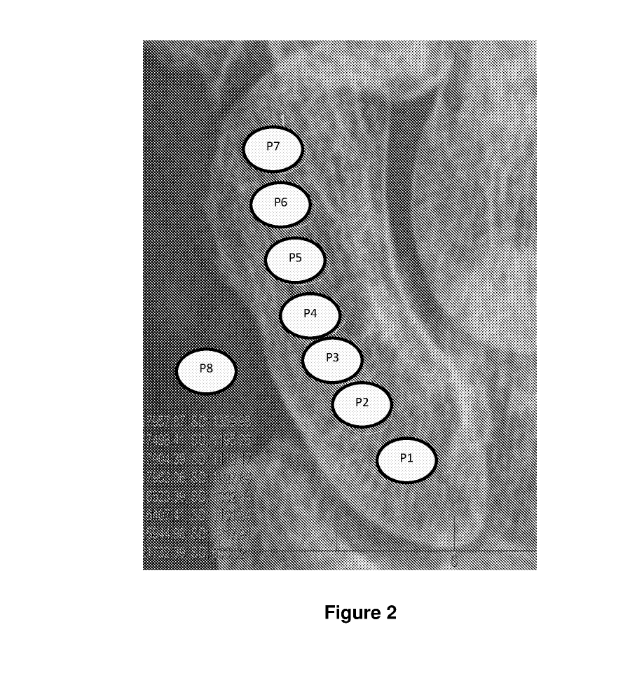

In a preferred embodiment, a plurality of measurement regions are selected along at least a portion of the bone 12, with a number of measurement regions selected on either side of the fracture 14. In the example shown in FIG. 2, where measurement regions are marked out substantially along the axis of a scaphoid bone from proximal to distal end, the plurality of measurement regions are conveniently referenced as P1-P7. The size and shape of the measurement regions can be varied to suit the particular application at hand. Typically, the measurement regions will each be less than or equal to the size of the portion of bone either side of the fracture 14.

In the example shown in FIG. 2, the measurement regions are marked out as small, substantially disc-shaped regions, each approximately 0.2 mm². However, it should be understood that size and shape of the measurement regions can be varied to suit the type and size of material being assessed. For example, in the assessment of a fracture of a larger bone, it is reasonable that each measurement region may be larger. Similarly, the overall number of measurement regions marked out on any image 10 may be adapted to suit the particular application. Again, in the assessment of a fracture to a larger bone, it is reasonable that a relatively greater number of measurement regions may be marked out on the image 10.

The measurement regions are marked out on the image 10 along the region corresponding to the bone 12 in an arrangement that substantially follows an axis based on any natural curvature of the bone 12. The measurement regions are each spaced apart from each other along this natural curvature. In the example shown in FIG. 2, the measurement regions are located at intervals whereby a central point of each measurement region is located 2 mm from the central point of the adjacent measurement region. That is, the measurement regions are spaced apart at 2 mm intervals. The distance between respective measurement regions may be varied in accordance with particular needs. Whereas a distance of 2 mm may be appropriate for a small bone such as the scaphoid, a greater distance may be more appropriate for assessment of a larger bone. This distance can therefore be varied as required.

It is preferred that at least one measurement region, a central measurement region, be marked on the image 10 substantially over the line of the fracture 14 or, in the case of a suspected fracture which is not readily visible, over the region that a fracture is suspected. In the assessment of a scaphoid bone, as shown in FIG. 2 the central measurement region is located on or about the waist of the scaphoid. In FIG. 2, the central measurement region is referenced as P4.

In addition to the aforementioned measurement regions, at least one reference measurement region 20a is selected and marked out on a portion of the image 10. In assessment of bone 12, the reference measurement region 20a is located on an area of the image 10 without bony shadows. In FIG. 1, the reference measurement region 20a (or P8 in FIG. 2) is marked out on a portion of the image 10 nearby the bone 12 and corresponding to a fleshy region of the body part from which the image 10 is taken. A reference measurement region 20b can alternatively or additionally be selected and marked out externally of the image 10, for example, on a separate image (not shown), this separate image ideally having been taken using the same imaging technology and specific imaging apparatus.

The reference measurement region or regions 20a, 20b are taken to take account for differences in, for example exposure of the image 10, particular specifications of the machine or apparatus used to take the image 10 and to eliminate effects of background ‘noise’ which all may affect measurements taken of regions of the image 10 corresponding to the bone 12. In this way, ‘noise’ created by, for example, fleshy portions of the body surrounding the bone 12 or of a cast surrounding the injured body part, can be accounted for in the subsequent calculations included in the method of the present invention.

A set of optical density (OD) measurements are taken of each measurement region and also of any reference measurement regions. The OD of each region is measured by suitable means, including using suitable software. For example, OD of each measurement region can be measured using the Region of Interest (ROI) tool on a picture archiving and communication system (PACS). It is preferred that an average OD measurement is taken for each region, typically the mean of at least three separate OD measurements for each region. These measurements are referred to as the raw OD values.

Each raw OD value corresponding to a measurement region of the bone 12 is then standardised relative to the reference measurement OD value to account for the aforementioned differences in exposure, machine specifications/variations and the like.

A series of standardised OD values corresponding to each measurement region of the bone 12 are determined from the difference between the reference measurement OD and each raw OD value corresponding to a measurement region. For example, referring to FIG. 2, the standardised OD value of P1 would be the OD of P8 minus the OD of P1. This calculation provides a series of standardised OD measurements, S1 to Sx where x is the total number of measurement regions from which an OD reading has been taken on the particular image 10. Referring again to FIG. 2, each OD measurement P1 to P7, once standardised, is then conveniently referenced as standardised OD values S1 to S7. These standardised OD values are standardised for variable exposure and penetration by calculation of the difference between the reference measurement region OD and each of the measurement region OD values.

Each standardised OD value is then normalised against an appropriately selected S value. Each standardised OD value is desirably standardised against a measurement taken at the outermost region of the portion of bone 12 being assessed on the image 10.

Referring to FIG. 2, OD measurements are taken along substantially the span of the natural curvature of the scaphoid. Each standardised OD value is normalised against a standardised OD measurement taken at or near the proximal pole of the scaphoid. In FIG. 2, this equates to the standardised OD value corresponding to P1, i.e. S1. Thus, each standardised OD value S2 to S7 is normalised by dividing by the value of S1. These normalised OD values are conveniently given the reference N and thus standardised OD values S1 to Sx yield values N1 to Nx. Again, x is the total number of measurement regions from which an OD reading has been taken on the particular image 10 being assessed. Thus, for the OD measurements taken from the image 10 of the scaphoid of FIG. 2, the normalised OD values are conveniently referenced as N1 to N7.

An OD gradient (G) or ratio is then calculated between every possible pairing of N1 to Nx. Referring again to FIG. 2, where there are normalised N values N1 to N7, a total of twenty one gradients are calculated from these pairings. The OD gradient is calculated as a function of the distance between the N values in every pair. While different mathematical functions may be usefully employed, in this embodiment we have used G_{i,j}=(Nᵢ−Nⱼ)/(distance between Nᵢ and Nⱼ) That is, OD gradient between the pairing of N1 and N2 (G1,2) can be calculated as: G1,2=(N2−N1)/Distance between N2 and N1).

Distance between Nᵢ and Nⱼ is measured with reference to the same location on each measured region. In this present example, distance is measured between the central point of each measurement region. However, distance could also be measured between respective proximal or distal sides of each measurement region. In the example given in FIG. 2, where the measurement regions and therefore the spatial arrangement of the normalised OD values N1 to N7 are spaced apart at 2 mm intervals (between the respective central points), the OD gradient G (G1) between the pairing of N1 and N2 can be calculated as: G1,2=(N2−N1)/0.2.

Referring to FIG. 1, the OD gradient between the normalised OD values of first measurement region 16 (N1) and second measurement region 18 (N2), having distance 22 (D) therebetween would be:

G1,2=(N2−N1)/D

In the present example, having measurements N1 to N7, a total of twenty one (21) OD gradients (G1 to G21) are determined. In assessment of the bone fracture to predict likelihood or occurrence of non-union, inventors have determined that the OD gradient between N1 and N7 (OD gradient of the proximal and distal OD measurements) provided best correlation of ability to predict. OD gradients of other pairings in this example demonstrated less correlation. However, in assessment of different types of bones or in utilising the present method to assess a different characteristic, OD gradients of other pairings may be most appropriate or most useful. For example, in determining when to remove a plaster cast from a patient, it is possible that one or more OD gradients other than the N1 to Nx pairing may be more appropriate.

Each of the calculations described above may conveniently be performed for each OD of each measurement region on any image 10 automatically. For example, it is reasonable that such calculations can be performed as a function of software used in determining the initial raw OD values. Such automation conveniently provides a rapid assessment of the OD gradient of an actual or suspected bone fracture, which may then be utilised in clinical assessment of the fracture.

OD gradient(s) is desirably initially determined on or shortly after injury leading to the fracture or suspected fracture. OD gradient(s) is also determined on at least one more occasion, some time after date of injury. The length of time after injury that OD gradient is determined varies depending on the bone being assessed, the type of fracture and for what purpose (e.g. prediction of non-union or union) the OD gradient is being determined. In the case of determining likelihood or occurrence of non-union of a scaphoid, OD gradient is determined on at least one occasion, preferably at least two, between 6 and 100 days following injury. It has been found that determining OD gradient prior to about day 6 following injury, the gradient is too weak to be useful in such an assessment.

The inventors have determined, after conducting OD measurements and calculations as described above on a series of (i) normal bones, (ii) fractured bones resulting in normal or healthy union; and (iii) fractured bones resulting in non-union, that the aforementioned OD gradients can be advantageously used to assess or predict the occurrence or likelihood of non-union of the fracture. It has been found that by periodically performing imaging on fractures and determining optical density gradients as described above, it can be possible to make an early determination of those who would benefit from surgical intervention. Presently, the practice whereby an increasing number of patients are operated on shortly after fracture to avoid risk of non-union results in overtreatment of many for purposes of avoiding under-treatment of a few. Determination of optical density gradients and assessment of fracture induced gradients as described above can advantageously be used to risk stratify patients and undertake surgical intervention only on those patients whose injuries are not healing satisfactorily, thereby significantly reducing the overall number of unnecessary operations.

The method of the present invention will now be described in more detail with reference to the following non-limiting example, analysing a specific bone type, namely the scaphoid, in accordance with the present invention.

### Example

### Analysis of X-Ray Images of Scaphoid

Some 53 x-ray images, taken of scaphoid bones of twenty six patients were analysed using the method of the present invention. Of these twenty six patients, twenty one had a fracture through the waist of their scaphoid. Of these twenty one, twelve of the fracture injuries resulted in a non-union and nine progressed to normal union. As part of the study, a further five x-ray images of normal scaphoids were used as controls. At least two images were taken from each patient, generally over a period from day zero (equating to day of injury and/or day first image was taken) up to about day 49 after injury or first image was taken.

Seven disc-shaped measurement regions, each measuring 0.2 mm² were marked out on the portion of each x-ray image corresponding to the scaphoid bone. The first seven of these measurement regions were located and distributed along the natural curvature of the scaphoid, spaced apart at regular intervals such that the central point of each disc-shaped measurement region was 2 mm away from each adjacent measurement region. The distribution of the seven measurement regions is as shown in FIG. 2, with the measurement regions being referenced as P1 to P7. P1 is located adjacent the proximal pole of the scaphoid and P4 is located substantially over the fracture site or, in those images of scaphoid demonstrating no fracture, P4 is located over the waist of the scaphoid. A reference measurement region, referenced on FIG. 2 as P8, was marked out on a region of the image just outside the scaphoid, on an area of the image exhibiting no bony shadow. The reference measurement region P8 was of substantially the same size and dimension as the disc-shaped measurement regions.

The average optical density (OD) of each of the measurement and reference regions was measured using the ROI (Region of Interest) tool on the PACS (picture archiving and communication system) viewer, giving a series of raw OD values. These raw OD values were standardised for variable exposure and penetration by calculating the difference between the OD of the reference measurement area and the OD of the seven measurement regions to give a series of standardised OD values, referenced as S1 to S7. The standardised OD values were normalised to the OD measurement of the measurement region located at the proximal pole of the scaphoid (P1 and after standardisation, S1) and these normalised OD values were sequentially referenced as N1 to N7.

An OD gradient or ratio was calculated for each pairing of N1 to N7 on each image, giving a total of twenty one OD gradient values for each image. The resulting twenty one OD gradient values of each image were compared in three groups: (i) normal scaphoids; (ii) fractured scaphoids resulting in healthy or normal union; and (iii) fractured scaphoids resulting in non-union.

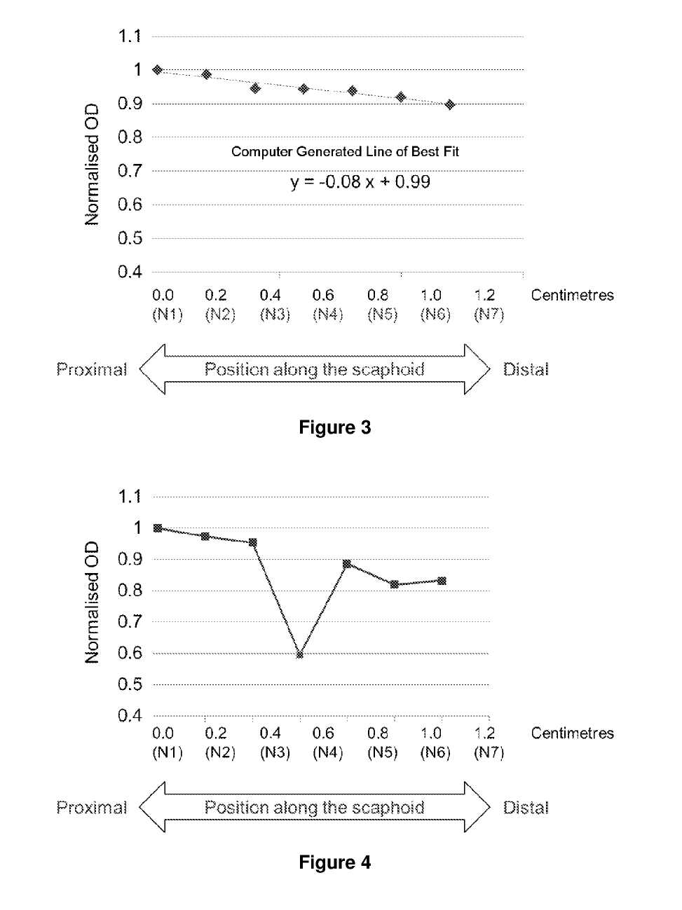

On analysis of the results of the OD gradients calculated by the method described above, it has been determined by the inventors that normal scaphoids having no fracture injury demonstrate an OD gradient that proceeds along the long axis of the scaphoid. Analysis of multiple images of normal, uninjured scaphoids demonstrated that these consistently display a reproducible gradient. Analysis of N values along the long axis of the scaphoid from proximal to distal pole demonstrates a consistently changing optical density from the proximal to distal pole having an average slope of about −10%. This is represented graphically in FIG. 3, where it can be seen that the OD gradient is substantially linear and has a slope of about −10%.

In contrast, images of scaphoids having a fracture site analysed as per the method described above, demonstrated a visibly different OD gradient. Instead of the substantially linear gradient derived from analysis of uninjured scaphoids, those with a fracture instead show an acute differentiation to the linear gradient at or near the fracture site.

Referring to FIG. 4, there is shown a graphical representation of the OD gradient calculated along a fractured scaphoid from the proximal to distal ends as per described above, where the fracture site is located adjacent the N4 OD measurement. From this analysis, it is shown that mapping of OD gradient demonstrates that fractures exhibit as acutely formed gradients. This acutely formed gradient is conveniently referred to as a fracture induced gradient.

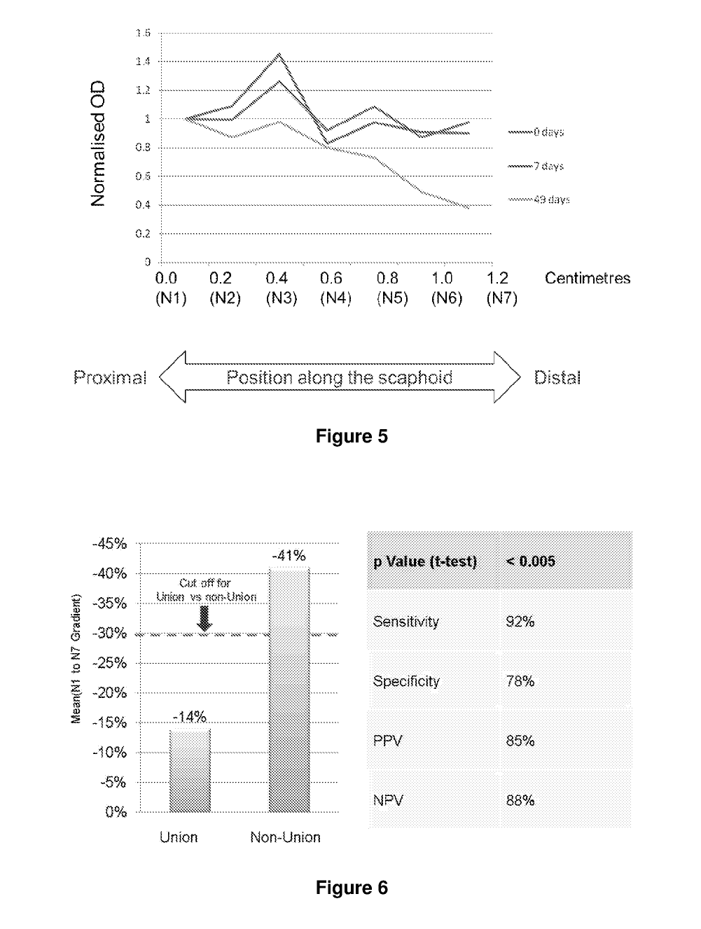

The OD gradient was similarly quantified for the multiple images taken from the same patient at different time intervals. One example of this is shown in FIG. 5, which illustrates the OD gradient of a fractured scaphoid, evaluated from the proximal to distal end as per described above and where the fracture site is located adjacent the N4 OD measurement. In FIG. 5, the OD gradient has been calculated at day zero (date of injury) and subsequently at 7 days and 49 days after injury.

In FIG. 5, it is seen that the fracture induced gradients vary over time, whereby the acutely formed gradient is effaced over time as normal fracture healing progresses. That is, the value of the fracture induced gradient varies over time as fracture healing progresses. It is therefore apparent from these results, that fracture induced gradient varies both spatially (along the axis of measurement of OD) and over time, demonstrating that fracture induced gradients are evolutionary gradients.

Referring to FIG. 5, it can be observed that the fracture induced gradient slopes upwardly, which contrasts to the downwardly sloping gradient illustrated in FIG. 4. In the first few hours and days following injury leading to fracture, the area around the fracture site undergoes intense inflammatory activity. It is proposed that this inflammatory activity leads to a rise in the normalised OD immediately adjacent to the fracture since inflammation results in an outpouring of cells and fluid from blood vessels into the area around the fracture. It is proposed that the fracture induced gradient at day zero in FIG. 5 is a consequence of this outpouring of cells and fluid.

Notably, it is proposed that it is logical that asymmetry will be observed in the fracture induced gradient across a fracture that is headed for non-union as one side of the fracture has normal blood supply and the other is avascular (without blood supply). In the present example, such asymmetry, correlating to occurrence of non-union of the fracture was observed. It is proposed that observation of such an asymmetry may provide an additional means of predicting likelihood or occurrence of non-union.

In the present example, the OD gradient of N1 to N7 was calculated for each image. In this example, where each image is of a scaphoid, each measurement region is 2 mm apart from the adjacent measurement region and a total of seven OD measurements are taken along the axis of the scaphoid from proximal to distal pole, the distance between N1 to N7 is 1.2 cm. The OD gradient (G1,7) of N1 to N7 is therefore calculated as:

G1,7=(N2−N1)/1.2

Analysis of the gradient of N1 to N7 as per the above equation of multiple images demonstrated that there are significant differences between fractures headed for union and those headed for non-union.

From the results obtained by analysing this sample of 53 images, it was determined that those images of fractures headed for non-union showed a mean gradient of −41%. In contrast, those headed for union showed a mean gradient of −14%. These differences were statistically significant with a p-value of 0.005. From these results, it is apparent that a steeper gradient or a gradient of a higher numerical value, is predictive of non-union of the fracture.

It was subsequently determined that a cut-off gradient value of −30% appropriately distinguishes between fractures headed for union and those headed for non-union. In this example, it was determined that utilising 30% as the cut-off provided highest sensitivity. This is illustrated in FIG. 6, where the sensitivity and specificity values are 92% and 78% respectively and positive predictive value (PPV) and negative predictive value (NPV) are each 85%.

From the above findings, it is proposed that taking periodic radiological images of actual or suspected fracture sites and quantification of OD gradient of these images at and around the fracture site, substantially as per the above example, can advantageously provide early identification of fractures at risk of non-union.

Early identification of those at risk of non-union advantageously facilitates early implementation of appropriate strategy to mitigate the non-union, thereby leading to faster and satisfactory fracture healing and cessation of periods of immobilisation as soon as possible.

It is apparent, from the analysis of images conducted in the above Example, that a fracture induced gradient forms at least within a few days following the fracture injury and, when fracture healing progresses normally and towards union, the fracture induced gradient value gradually falls over time.

Periodic analysis of radiologic images according to the present invention therefore provides a means of assessing whether fracture healing is progressing adequately. It also provides a means of defining, as the value of the fracture induced gradient falls, an appropriate point whereby the value of the fracture induced gradient indicates when a clinically acceptable stage of fracture healing has been achieved. In the scaphoid example, this may equate to, for example, a peak fracture induced gradient of less than 10%.

It is proposed also that analysis of radiologic images as per the method of the present invention may be used in estimating a date for adequate fracture healing for a particular injury of a particular patient. That is, analysis of radiologic images can be utilised to predict the amount of time it will take for the fracture induced gradient to fall below a critical minimum, the critical minimum equating to the clinically acceptable stage of fracture healing. Such capacity to project healing time advantageously provides patients with an individualised estimate of when their fracture is likely to have adequately healed, allowing for more effective planning and time management.

It is additionally proposed that the present invention finds application in diagnosis and assessment of bone disorders or disease, such as osteoporosis. The method may be usefully applied in diagnosing local and/or systemic osteoporosis, desirably before the disease has become established in a patient. In one example, the technique of the present invention can be used in detection of exaggerated normalised optical density values compared to similarly measured values from a ‘normal’ subject. In assessment for osteoporosis, this ‘normal’ bone is typically a comparison in density of a normal, healthy adult of about 30 years of age. The onset of osteoporosis within any given bone is typically not homogenous throughout the entirety of the bone. Therefore, exaggerated normalised values and/or gradients in a suspected bone, as measured by the method of the present application, can be indicative of presence or onset of osteoporosis. Advantageously, the present method can detect presence of both systemic and localised osteoporosis. This is an improvement over current diagnostics using DXA, where typically only a representative portion of a particular bone, usually hip bone, is measured. Current methods may therefore miss detection of localised osteoporosis.

Further, since progression of osteoporosis typically induces micro-fractures within bones, the method can be used, essentially as described above, to detect these micro-fractures which may otherwise normally be invisible by routine imaging techniques. Identification of such micro-fractures, which are themselves indicators of onset of osteoporosis, can therefore provide a useful early screening means for detection of osteoporosis, ideally before the patient presents with any serious fracture injury. The present invention therefore finds application in screening for bone disorders and diseases such as osteoporosis.

It is known that osteoporosis can affect different bones at different rates. The present method therefore finds application in determining optical density differences and gradients between regions in different bones to make an assessment of or give indication of different rate of onset or affliction of osteoporosis in these bones. As an example, it may be desired to determine variation of onset of the disease in lumbar vertebrae and lower limb bones. In such an application, the first region corresponds to a portion of a lumbar vertebrae and the second region corresponds to a portion of a lower limb bone. In use of the method in making such a diagnosis, the first and second regions and any other measured regions will ideally correspond to a volume of the respective bones.

Modifications and variations to the method described in this specification as may be apparent to the skilled reader are deemed to be within the scope of the present invention.

1. A method of assessing an optical density gradient in one or more radiologic images of a non-uniformly composed material, the method comprising:
a) determining optical density of at least a first region and a second region of the one or more radiologic image;
b) determining distance between the first and second regions,
c) calculating the optical density gradient as a function of the difference in optical densities of the first and the second regions and the distance therebetween; and
d) using the optical density gradient to assess a characteristic of the non-uniformly composed material.

2. The method of claim 1, wherein the radiologic image is an image taken using imaging technology capable of exhibiting variations in density.

3. The method of claim 2, wherein the imaging technology is capable of exhibiting variations in optical density.

4. The method of claim 1, wherein the radiologic image is one of an x-ray, computed tomography, magnetic resonance, ultrasound or positron emission tomography image.

5. The method of claim 1, wherein optical density is determined for a plurality of regions on the image corresponding to the non-uniformly composed material.

6. The method of claim 1, wherein each region for which optical density is to be determined is marked out on the image and size and shape of each region are determined with reference to the nature of the non-uniformly composed material and/or the characteristic of the non-uniformly composed material that is being assessed.

7. The method of claim 6 wherein each region is marked out on the image at substantially regular intervals along at least one predetermined axis of a portion of the material.

8. The method of claim 1, wherein optical density is determined for a reference region, optical density of the reference region being used to standardise optical density values of other measured regions.

9. The method of claim 8 wherein standardised optical density values are normalised against a pre-selected standardised optical density measurement.

10. The method of claim 9 wherein the standardised optical density values are normalised against a standardised optical density value of the first region to give normalised optical density values (N).

11. The method of claim 10 wherein the normalised optical density values are utilised to determine one or more optical density gradients (G).

12. The method of claim 10 wherein an optical density gradient is calculated between every possible pairing of N values for the image.

13. The method of claim 10 wherein the optical density gradient (G) between a pair of N values is represented by:
G_{i,j}=(Nᵢ−Nⱼ)/(Distance between Nᵢ and Nⱼ)
where i≠j and i, j are any numbers ranging from 1 to x and where x is the total number of measurement regions from which an optical density reading has been taken on the image being assessed.

14. A method of predicting occurrence or likelihood of non-union of a bone fracture, the method comprising:
a) determining optical density of at least a first region and a second region of a radiologic image corresponding to the bone;
b) determining distance between the first and second regions;
c) determining an optical density gradient between the first and second regions as a function of difference in optical density between the first and second regions and the distance therebetween;
whereby an optical density gradient greater than a predetermined value is deemed indicative of likelihood or occurrence of non-union of the fracture

15. The method of claim 14 wherein the predetermined value is determined at least partially on the basis of one or more variables of the bone being assessed.

16. The method of claim 14 wherein optical density gradient relating to the fracture is determined over a period of time.

17. The method of claim 16 wherein a first optical density gradient is determined on or shortly after injury leading to the fracture and a second optical density gradient is determined at a predetermined time thereafter.

18. The method of claim 16 wherein variation in the optical density gradient over time is used to make a prediction of when the fracture has substantially reached or will reach a clinically accepted point of adequate healing.

19. The method of claim 18 wherein the fracture is deemed to have substantially reached a clinically accepted point of adequate healing once the optical density value falls below a predetermined minimum value.

20. The method of claim 14, whereby optical density readings are standardised with reference to a reference area on the image.

21. The method of claim 20 wherein the standardised optical density readings are normalised as a function of the standardised optical density values.

22. The method of claim 20, wherein the standardised optical density readings are normalised against a standardised optical density measurement corresponding to the first region.

23. A method of predicting occurrence or likelihood of a disorder or disease of bone, the method comprising:
a) determining optical density of at least a first region and a second region of at least one radiologic image corresponding to bone in a subject;
b) determining distance between the first and second regions;
c) determining an optical density gradient between the first and second regions as a function of difference in optical density between the first and second regions and the distance therebetween;
whereby an optical density greater than a predetermined value is deemed indicative of likelihood or occurrence of a bone disorder or disease.

24. The method of claim 23, wherein optical density readings are standardised with reference to an area on the image.

25. The method of claim 24, wherein the standardised optical density readings are normalised as a function of the standardised optical density values.

26. The method of claim 24, wherein the standardised optical density readings are normalised against a standardised optical density measurement corresponding to the first region.

27. The method of claim 20, wherein the bone disorder or disease is osteoporosis.

28. The method of claim 20, wherein the first and second regions correspond to a volume of bone.

29. The method of claim 20, wherein the first region corresponds to a portion of a first bone and the second region corresponds to a portion of a second bone.
